(19) United States
(12) Patent Application Publication
(10) Pub. No.: US 2016/0058335 A1
(43) Pub. Date: Mar. 3, 2016

(54) Sensor Incorporated into an Exercise Garment

(71) Applicant: ICON Health & Fitness, Inc., Logan, UT (US)

(72) Inventor: Darren C. Ashby, Richmond, UT (US)

(21) Appl. No.: 14/838,041

(22) Filed: Aug. 27, 2015

Related U.S. Application Data
(60) Provisional application No. 62/044,021, filed on Aug. 29, 2014.

(51) Int. Cl. A61B 5/11; A61B 5/0488; A61B 5/00

(52) U.S. Cl. CPC A61B 5/1118; A61B 5/4866; A61B 5/0488; A61B 5/7455; A61B 5/6807

(57) ABSTRACT

A garment includes at least one electrode attached to a fabric of the garment, and at least one accelerometer attached to the fabric of the garment. A wireless device in is communication with an activity information device, and at least one haptic input mechanism is incorporated into the fabric that provide a haptic response based on communications with the activity information device.

### RELATED APPLICATIONS

This application claims priority to U.S. Patent Application Ser. No. 62/044,021 titled “A Sensor Incorporated into an Exercise Garment” and filed on 29 Aug. 2014, which application is herein incorporated by reference for all that it discloses.

### BACKGROUND

While numerous exercise activities exist that one may participate in, exercise may be broadly broken into the categories of aerobic exercise and anaerobic exercise. Aerobic exercise generally refers to activities that substantially increase the heart rate and respiration of the exerciser for an extended period of time. This type of exercise is generally directed to enhancing cardiovascular performance. This exercise usually includes low or moderate resistance to the movement of the individual. For example, aerobic exercise includes activities such as walking, running, jogging, swimming or bicycling for extended distances and extended periods of time.

Anaerobic exercise generally refers to exercise that strengthens skeletal muscles and usually involves the flexing or contraction of targeted muscles through significant exertion during a relatively short period of time and/or through a relatively small number of repetitions. For example, anaerobic exercise includes activities such as weight training, push-ups, sit-ups, pull-ups, or a series of short sprints.

Often, an athlete attempting to reach fitness goals desires to track the performance of his or her workout. Activity trackers are devices that can record some aspects of the user's workout. One type of activity tracker is a pedometer, which is a wearable device that tracks the steps that a user takes over time. In some instances, the pedometer is attached to a person's belt or another location on the user's body or clothing. As the user takes a step, an accelerometer or pendulum integrated into the pedometer senses the user's movement associated with the step and increments a counter that tracks the number of steps. In addition to pedometers, other types of activity trackers can be worn around a user's wrist. Some of these activity trackers share similarities with the pedometer. For example, such activity trackers can count the movement of the user's arm as well as the movements that are related to the user's steps. These devices can provide feedback to the user about his or her activities.

One type of system that provides feedback on a user's activity is disclosed in U.S. Patent Publication No. 2013/0086729 issued to Karin E. Carter, et al. In this reference, articles of apparel include a garment structure having one or more fabric elements structured and arranged to provide a close fit to at least one predetermined portion of a body (e.g., area(s) of the body for which enhanced position sensing and/or feedback are desired); and a body position feedback system engaged with or integrally formed as part of the garment structure. The body position feedback system may apply higher tensile or constricting (compressive) forces to selected portions of the wearer's body and/or stretch resistance, which can help stimulate or interact with nerves and deep tissue receptors located in various portions of the body. The increased forces at selected locations of the body give the wearer sensory feedback regarding the position or orientation of these parts of the body and can improve or accelerate development of muscle memory. Other types of systems are described in U.S. Pat. No. 7,072,721 issued to Cecilio Trent and U.S. Pat. No. 5,368,042 issued to John L. O'Neal. Each of these references are herein incorporated by reference for all that they disclose.

### SUMMARY

In one embodiment, a garment includes fabric, at least one electrode attached to the fabric, at least one accelerometer attached to the fabric, a wireless device in communication with an activity information device, and at least one mechanism incorporated into the fabric that provides a haptic response in response to communications with the activity information device.

The activity information device may be a fitness tracking device.

The activity information device may be an environment simulation device.

The environment simulation device may be programmed to cause an aerobic exercise device to simulate a real world route.

The environment simulation device may be programmed to simulate a sporting event.

The at least one electrode may be part of a surface electromyography system.

The at least one electrode may be interwoven into the fabric.

The garment may include an accelerometer is interwoven into the fabric.

The at least one electrode may measure muscle activity of a user wearing the garment when the garment is worn by a user.

The at least one electrode may include multiple electrodes positioned to measure activity of at least two of a forearm muscle, a bicep muscle, a shoulder muscle, and a tricep muscle group.

The at least one electrode may include multiple electrodes positioned to measure activity of at least two of a quadriceps muscle, a hamstring muscle, an adductor muscle, and a calf muscle.

The at least one electrode may include multiple electrodes positioned to measure activity of at least one of a shoulder muscle, a trapezius muscle, and a latissimus dorsi muscle.

The at least one electrode may include multiple electrodes positioned to measure activity of at least one of a pectoralis muscle, an abdominal muscle, and an oblique muscle.

The activity information device may determine a force generated by a user during a workout.

The haptic input mechanism includes an eccentric weight and a motor.

In one embodiment, a garment includes fabric, at least one electrode interwoven into the fabric where the at least one electrode is part of a surface electromyography system, at least one accelerometer attached to the fabric, and a wireless device in communication with an environment simulation device, wherein the at least one electrode provides a haptic response based on communications with the environment simulation device.

The environment simulation device may be also a fitness tracking device.

The fitness tracking device may determine a number of calories burned by a user during a workout.

The garment may also include a location determining mechanism.

The garment may be a sock.

In one embodiment, a garment includes compressive fabric, at least one electrode interwoven into the compressive fabric where the at least one electrode is part of a surface electromyography system, at least one accelerometer attached to the compressive fabric, and a wireless device in communication with a device that simulates a workout environment and tracks fitness of a user. The at least one electrode provides a haptic response based on user interaction with a simulated object from the workout environment with the device, and the wireless device determines a force generated by the user during a workout and determines a number of calories burned by the user during the workout.

### BRIEF DESCRIPTION OF THE DRAWINGS

The accompanying drawings illustrate various embodiments of the present apparatus and are a part of the specification. The illustrated embodiments are merely examples of the present apparatus and do not limit the scope thereof.

FIG. 1 illustrates a perspective view of an example of a garment in communication with a computing device in accordance with the present disclosure.

FIG. 2 illustrates a block diagram of an example of an activity system in accordance with the present disclosure.

FIG. 3 illustrates a view of an example of a display incorporated into a computing device in accordance with the present disclosure.

FIG. 4 illustrates a perspective view of an example of a garment tracking a user activity in accordance with the present disclosure.

FIG. 5 illustrates a perspective view of an example of a garment tracking a user activity in accordance with the present disclosure.

Throughout the drawings, identical reference numbers designate similar, but not necessarily identical, elements.

### DETAILED DESCRIPTION

The principles described in the present disclosure include a garment that includes at least one electrode attached to a compressive fabric of the garment. The garment may also include an accelerometer attached to the fabric and have a wireless device 106 in communication with a fitness information device. At least one mechanism is incorporated into the fabric that is configured to provide a haptic response to the user based on communications with the fitness information device.

The electrode, accelerometer, location determining device, and other sensors incorporated into the garment can be used to gather information about the activities performed by the user. Such information may track the types of movements performed by the user, the number of movements performed by the user, the heart rate of the user, the oxygen level of the user, the muscle stimulation of the user, other types of information about the user, and combinations thereof. This information may be used to determine fitness data about the user's workout. For example, the information may be sent back to the user in real time to provide feedback about the user's workout. For example, if the user has a goal to burn a predetermined number of calories during a workout, the sensors in the garment may collect sufficient types of data to provide an estimated or calculated calorie burn to help the user determine whether the user has met his or her goal. In another example, the user may desire to keep his or her heart rate below a predetermined level. The feedback from the sensors in the garment may help the user understand whether or not he or she is exceeding his or her predetermined heart rate level.

The garment may be any appropriate type of garment. For example, the garment may be a shirt, shorts, pants, socks, other types of garments, or combinations thereof. The garments may be appropriate for any type of activity. For example, the sensors may collect data about the user during anaerobic exercises, aerobic exercises, or combinations thereof. Further, a user's activity may include playing basketball, dodge ball, baseball, football, capture the flag, volleyball, badminton, tennis, softball, soccer, hockey, water polo, other activities, or combinations thereof. Further, a user activity may include other active activities often performed for recreational purposes such as wrestling, fencing, curling, skiing, running, walking, swimming, stretching, rowing, dancing, golfing, horseback riding, kayaking, skateboarding, snorkeling, diving, staking, scuba diving, rock climbing, boxing, snow shoeing, martial arts, yoga, other activities, or combinations thereof. For the purposes of this disclosure, the user's activity may also include daily activities such as gardening, shoveling, watching television, sleeping, standing in line, reading, eating, playing with kids, cleaning, cooking, shopping, performing home repairs, other types of activities, or combinations thereof. While some of these activities are not physically intense, the sensors in the garment can nevertheless gather information about the exertion by the user in performing these activities.

In some examples, the electrode in the garment is at least part of the mechanism that provides a haptic response to the user. For example, the haptic response may be in response to reaching an undesirable health condition. For example, if the user is exceeding a desired heart rate, the electrode may provide a haptic response to indicate to the user his or her condition. In other examples, the haptic response is part of a simulation. For example, if the user is playing tennis with a virtual player depicted on a screen, the haptic response may occur in response to a virtual ball hitting the user to make the simulation appear more real. Further, the haptic response may occur in a simulation where a user is hit with a virtual tree branch as the user is running through a simulated forest.

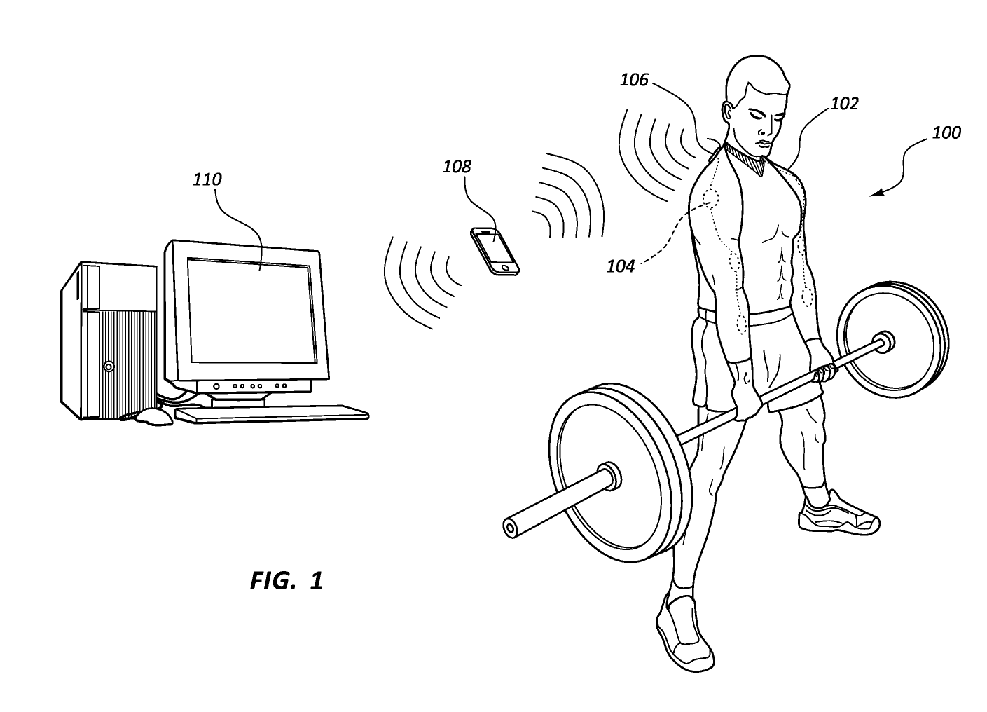

Particularly, with reference to the figures, FIG. 1 depicts a user 100 wearing a garment 102 with at least one sensor 104 incorporated into the garment's fabric. In some examples, a wireless device 106 is incorporated into the garment's fabric which can send and receive signals from other sources. The wireless device 106 may be in communication with an activity information device, such as a fitness tracking device, that tracks the information gathered by the sensors 104 of the garment 102. The activity information device may be a mobile device 108, a laptop, a desktop, a cloud based device, a storage device, a digital device, another type of device, or combinations thereof. In the example of FIG. 1, the activity information device is a desktop 110 that is in indirect communication with the garment 102 through the mobile device 108.

The sensors 104 may include at least one electrode that is capable of detecting at least one characteristic of the user. For example, one or more electrodes may be positioned adjacent regions of the user's body through the garment 102 to receive electrical cardio signals of the user. The electrical cardio signals can be used to determine the user's heart rate.

In another example, an electrode may be positioned to receive electromyography signals that detect muscle contraction. For example, the sensors 104 may be positioned over each muscle group of interest. In the example of FIG. 1, sensors 104 are positioned over the user's deltoid muscles, bicep muscles, and forearm muscles. But, the surface electromyography sensors can be positioned proximate pectoral muscles, trapezius muscles, oblique muscles, abdominal muscles, latissimus dorsi muscles, tricep muscles, hamstring muscles, quadriceps muscles, calf muscles, adductor muscles, other types of muscles, or combinations thereof. As the muscles contract, the corresponding electromyography sensor may detect an electrical signal indicating the muscle contraction. In some examples, the electromyography sensor can also detect the contraction percentage of the muscle. The contraction percentage can indicate to the user how effectively he or she is working out his or her muscles.

The garment 102 may also include accelerometers. The accelerometers may be incorporated into the garment 102 in any appropriate location to determine the types of body movements performed by the user. For example, a three axis accelerometer may be incorporated into the garment 102 to determine vertical and horizontal movements. The movement patterns can be analyzed to determine the user's types of movements. For example, jumping movements may exhibit different types of patterns than walking movements. Further, one or more accelerometers may be incorporated into each sleeve of the garment 102. For example, an accelerometer incorporated into the upper portion of the sleeve can detect when the upper arm moves, and another accelerometer incorporated into the lower sleeve can detect when the lower arm moves. With this arrangement, complex arm movements can be detected. Similarly, other arrangements with multiple accelerometers can determine whether the user is jumping, twisting, curling, walking, running, performing another type of movement, or combinations thereof.

Accelerometers may also be used to determine a respiration count of the user. For example, at least one accelerometer positioned about the user's chest can be used to determine when the user's chest expands and contracts in accordance with the user's breathing. In other examples, a strain gauge may be incorporated into the garment 102, and as the user's chest expands from breathing, the strain gauge stretches. As the strain gauge stretches, it generates a signal that can be sent to the activity information device.

Such recorded movements, in conjunction with recorded muscle contractions, provide a degree of detail about the user's workout. These details can be analyzed to determine the force exerted by the user during the workout, the number of calories burned by the user during the workout, other types of details about the workout, or combinations thereof.

In some examples, the garment includes a location determining device. This device may be a global positioning device, another type of location determining, or combinations thereof. In some examples, the location determining device may be used to determine a distance that a user has traveled, a speed at which the user has traveled, an altitude at which the user is at, an altitude that the user has traveled, other parameters, or combinations thereof.

Each sensor may be in communication with the wireless device 106. In some examples, each sensor is in communication with the wireless device 106 through an independent electrically conductive medium. In other examples, the sensors 104 communicate with each other and communicate with the wireless device 106 through other sensors 104. For example, the wireless device 106 may be in direct communication with a first sensor and indirectly in communication with a second sensor through the first sensor. The second sensor can send information towards the wireless device 106 by sending the information to the first sensor, which then sends the information on to the wireless device 106. In this example, the sensors 104 form a network. This sensor network may allow sensors 104 to communicate with each other. In some examples, these communications are bidirectional where the first sensor can send messages to the second sensor and the second sensor can send messages to the first sensor. These networks may have any appropriate network topology, such as a daisy chain topology, a bus topology, a star topology, a mesh topology, ring topology, a tree topology, a linear topology, a fully connected topology, another type of topology, or combinations thereof.

An electrically conductive medium may include a cable or another type of wire that is disposed within channels formed within the fabric of the garment 102. In other examples, an electrically conductive thread is used to create an electrically conductive pathway formed in the fabric of the garment 102. For example, a single thread may be used to create the electrically conductive pathway. In other examples, multiple threads are used to form a patch of electrically conductive fabric capable of conducting an electrical signal. This electrically conductive fabric may be covered by an outer fabric layer, an inner fabric layer, a waterproof layer, a breathable layer, another type of layer, or combinations thereof. In some examples, an electrically conductive fabric is exposed in the inner or outer surfaces of the garment 102.

While the above examples have been described with the sensors 104 being in communication with each other or with the wireless device 106, any appropriate communication mechanisms may be used to enable communication between the components of the garment 102. For example, the sensors 104 may be in communication with each other through fiber optic cables, wireless transceivers, other types of communication channels, or combinations thereof. In some examples, the garment 102 includes multiple wireless devices that are capable of communication with the activity information device directly or indirectly.

In some examples, the activity information device is caused to record and store the information received from the garment 102 by an activity tracking program. One type of activity tracking program that may be compatible with the principles described in the present disclosure is the iFit program which is accessible through website www.ifit.com (last visited May 19, 2014) and administered through ICON Health and Fitness, Inc. headquartered in Logan, Utah. The activity tracking program may calculate the number of calories burned by the user, track the amount of weight lifted by the user, track the number of sets and/or repetitions lifted by the user, determine the heart rate of the user, determine the respiration of the user, track the duration of the user's workout, record other types of information, or combinations thereof. In some examples, the user has access to the information about the workout in real time. In this examples, the information may be presented to the user, friend, trainer, or another type of individual to the user through a screen of the mobile device 108 or a larger screen that is easy/convenient for the user to view while working out. Further, the information may be available to the user after the workout is over. In some examples, the user may access the information over the internet. In other examples, the user may access the information through the local memory of the mobile device 108, laptop, desktop, or other digital device that contains the information. The information may also be available to other users of the activity tracking program. In these situations, the user may be in a competition with other users of the activity tracking program, or the user may allow a trainer, health professional, or other type of user to review the information.

In some examples, the activity tracking program gives the user advice. For example, if the user indicates that the user desires to achieve certain fitness goals within a predetermined time period, the activity tracking program may provide feedback to the user about his or her progress towards to the fitness goals. In some examples, the activity tracking program may give recommendations to the user to help assist the user towards his or her goals. These recommendations may include recommending that the user lift heavier weights for certain types of lifts, run at a faster pace, exercise for a longer period of time, other types of recommendations, or combinations thereof. In some examples, these recommendations may include safety recommendations, such as recommending to the user to slow down, lift lighter weights for specific lifts, other types of recommendations, or combinations thereof. These recommendations may be based in part of the user's heart rate, desired goals, the muscle percentage contraction, other types of information, or combinations thereof. While this example has been described as being compatible with a specific activity tracking program with specific features, any appropriate type of activity tracking program and/or features of this program may be used in accordance with the principles described herein.

In addition to collecting information about the user's activities and resulting physiological conditions, the sensors 104 may be configured to provide a haptic response to the user. These haptic responses may be generated for any appropriate type of reason. For example, a haptic response may be provided to indicate to the user that he or she is over exerting himself or herself. The haptic response may be generated by applying a safe localized stimulus to the user. This electrode may be used to gather specific types of data in addition to generating haptic responses. But, in other examples, the electrode may be dedicated to providing haptic responses. Haptic responses that are generated as a warning to the user may be accompanied with warnings displayed in the mobile device, an audible warning, or another type of warning mechanism. But, in some examples, the haptic response is the sole mechanism for warning the user about physiological condition.

The haptic response may also be administered as part of a simulation. For example, if the user is performing a workout that is simulated to be in a particular environment, such as a forest, and the user runs into a virtual obstacle, such as a tree, a haptic response may be administered to provide a realistic element to the simulation.

In other examples, a haptic response may be administered to assist the user in performing a lift. In some circumstances, an appropriately applied electrical pulse from an electrode may cause or increase a muscle contraction. Thus, if the user is struggling to lift a weight, a haptic response may be applied to cause the user's muscles to contract to complete the lift. The assisted contractions may contribute to increased muscles simulation and growth. In some examples, the user may have an option to have the haptic response assistance just when the user is struggling, for every lift, or not at all.

Further, while the above examples have been described with reference to performing calculations and other forms of interpreting the data collected by the sensors 104 with the activity information device, any appropriate location for performing these calculations and/or interpretations may be used in accordance with the principles described in the present disclosure. For example, this processing may occur on the mobile device 108, a networked device, a computing device incorporated into the garment 102, another type of device, or combinations thereof.

In some examples, a battery or another type of power source is incorporated into the garment 102. This battery may be a disposable battery or a rechargeable battery. In some cases, the garment 102 may include an energy harvesting mechanism, such as a linear generator that can harvest the movements of the user to produce energy or a thermoelectric device that can use the thermal differential between the user's body heat and the ambient temperature of the air surrounding the user to provide energy to power the sensors 104 of the garment. In some examples, the energy harvesting mechanisms supplement the battery or other power source in the garment 102 or the energy harvesting mechanism can be used to recharge the batteries.

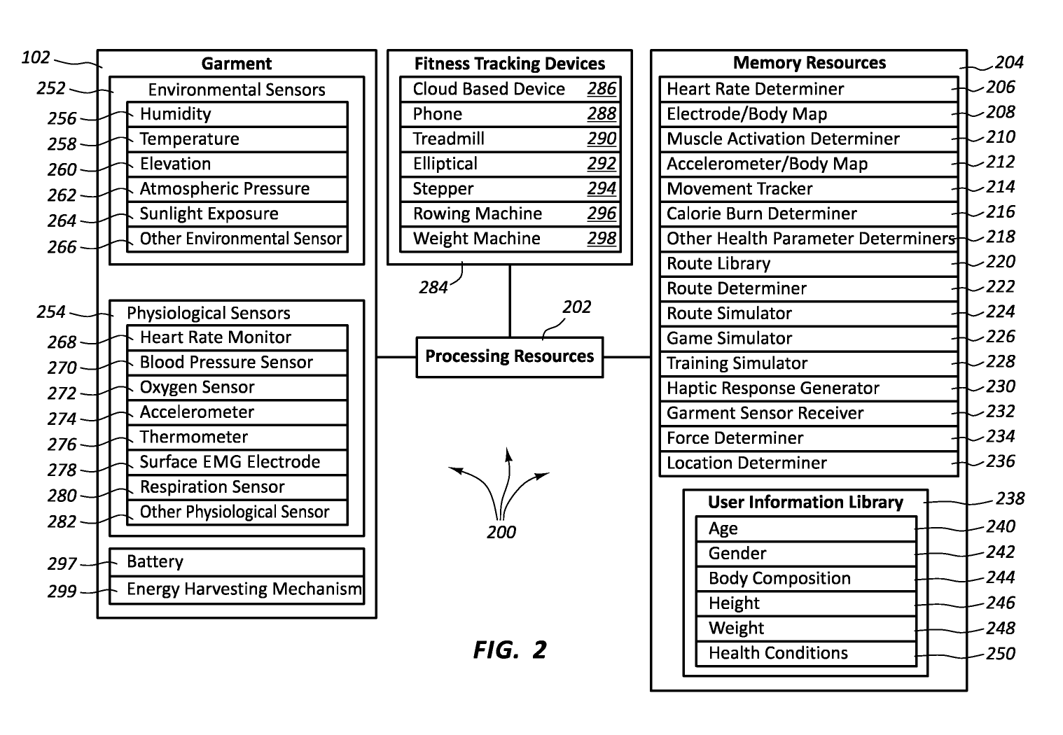

FIG. 2 illustrates a block diagram of an example of an activity system 200. The system 200 may include a combination of hardware and program instructions for executing the functions of the system 200. In this example, the system 200 includes processing resources 202 that are in communication with memory resources 204. Processing resources 202 include at least one processor and other resources used to process programmed instructions. The memory resources 204 represent generally any memory capable of storing data such as programmed instructions or data structures used by the system 200. The programmed instructions shown stored in the memory resources 204 include a heart rate determiner 206, a muscle activation determiner 210, a movement tracker 214, a calorie burn determiner 216, other health parameters determiner 218, a route determiner 222, a route simulator 224, a game simulator 226, a training simulator 228, a haptic response generator 230, a garment sensor receiver 232, a force determiner 234, and a location determiner 236. The data structures shown stored in the memory resources 204 include an electrode/body map 208, an accelerometer/body map 212, a route library 220, and a user information library 238.

The user information library 238 may include information that is specific to the user. For example, the personal information may include age information 240, gender information 242, body composition information 244, height information 246, weight information 248, and user health conditions 250.

In some examples, the garment 102 includes environment sensors 252 and physiological sensors 254. The environment sensors 252 may include humidity sensors 256, temperature sensors 258, elevation sensors 260, atmospheric pressure sensors 262, sunlight exposure sensors 264, and other environmental sensors 266. The sensors may be used to determine parameters of the user's workout. The indicators from the environment sensors 252 may assist in determining whether the user's workout was more difficult due to weather or other environmental conditions. The physiological sensors 254 may include heart rate monitors 268, blood pressure sensors 270, oxygen sensors 272, accelerometers 274, thermometers 276, surface electromyography electrodes 278, respiration sensors 280, other physiological sensors 282, or combinations thereof. The garment 102 may also include a battery 297 and/or an energy harvesting mechanism 299.

In some examples, the workout environment information may be gathered through another source other than through sensors incorporated into the garment 102. For example, the workout environment information may be accessed through a weather station, a map, a home climate system, a website, another type of information source, or combinations thereof.

The processing resources 202 may also be in communication with at least one fitness tracking device 284. In some examples, the fitness tracking device 284 is incorporated into a cloud based device 286, a phone 288, a treadmill 290, an elliptical 292, a stepper machine 294, a rowing machine 296, a weight machine 298, another type of exercise machine, another type of fitness accessory, or combinations thereof. Any of these devices may store and/or process information gathered by the sensors of the garment 102.

The processing resources 202, memory resources 204, garment 102, and other devices may communicate over any appropriate network and/or protocol. For example, these devices may be capable of communicating using the ZigBee protocol, Z-Wave protocol, BlueTooth protocol, Wi-Fi protocol, Global System for Mobile Communications (GSM) standard, another standard, or combinations thereof.

The memory resources 204 include a computer readable storage medium that contains computer readable program code to cause tasks to be executed by the processing resources 202. The computer readable storage medium may be a tangible and/or non-transitory storage medium. The computer readable storage medium may be any appropriate storage medium that is not a transmission storage medium. A non-exhaustive list of computer readable storage medium types includes non-volatile memory, volatile memory, random access memory, write only memory, flash memory, electrically erasable program read only memory, magnetic based memory, other types of memory, or combinations thereof.

The heart rate determiner 206 represents programmed instructions that, when executed, cause the processing resources 202 to determine the heart rate of the user. In some examples, the heart rate determiner 206 bases its determination on the recordings of the heart rate monitor 268. The muscle activation determiner 210 represents programmed instructions that, when executed, cause the processing resources 202 to determine which muscles are being activated by the user. In some examples, the muscle activation determiner 210 determines which electrode is receiving a signal, and the muscle activation determiner 210 consults with the electrode/body map 208 where each electrode is associated with a part of the body and/or muscle group. In this example, if an electrode with a serial number 0001 is placed over the user's right bicep, the electrode/body map 208 may associate electrode 0001 with the user's right bicep. When electrode 0001 receives a signal, the muscle activation determiner 210 may determine that the user's right bicep is being activated.

The movement tracker 214 represents programmed instructions that, when executed, cause the processing resources 202 to track the movement of the user. The movement tracker 214 may consult the accelerometer/body map 212 to determine the location of each accelerometer 274 incorporated into the garment 102. For example, if the accelerometer location on the user's lower right arm records that it is moving in an upward vertical direction, but an accelerometer located on the user's upper arm does not record any vertical upward movement, the movement tracker 214 may determine that the upper arm is remaining stationary while the lower arm is bending upwards. Further, the movement tracker 214 may also analyze patterns recorded by the accelerometers 274. For example, some movement patterns may indicate that the user is walking while other patterns indicate that the user is running By recognizing these patterns, the processing resources can determine which types of activity the user is performing.

The calorie burn determiner 216 represents programmed instructions that, when executed, cause the processing resources 202 to determine the number of calories that the user has burned for a specific period of time. This time period may coincide with the time period that the user is working out. The calorie burn determination may be based in part on the user's movement, heart rate, muscle activity, body weight, other factors, and/or combinations thereof.

The other health parameters determiner 218 represents programmed instructions that, when executed, cause the processing resources 202 to determine other health conditions of the user. The other health conditions may monitor the user's respiration, blood sugar characteristics, blood pressure, other blood characteristics, skin color, hydration levels, cramping, other health conditions, or combinations thereof.

In some situations, the garment 102 is used in conjunction with a workout that is incorporated into a simulation. For example, the simulation may be a course simulated on a treadmill, a stationary bike, an elliptical, a rowing machine, or another type of exercise machine. In these simulations, a screen may depict the scenery of the simulated course. In some cases, speakers may depict the sounds associated with the simulated route. Further, some simulations may include a depiction of the actual weather occurring in real time at the location that is being simulated. Also, the exercise machine, such as the treadmill or stationary bicycle, may change incline angle, tilt angles, speed, resistance, or other characteristics based on the characteristics of the simulated course. For example, if the simulated course includes an uphill portion, the treadmill may increase its incline as the user runs on simulated uphill portion of the course.

The route determiner 222 represents programmed instructions that, when executed, cause the processing resources 202 to determine which route to simulate. In some cases, the route is preselected from the route library 220. In other cases, the route is constructed by the user based on the user's input of geographic location using an online map. The route simulator 224 represents programmed instructions that, when executed, cause the processing resources 202 to simulate the selected route.

The game simulator 226 represents programmed instructions that, when executed, cause the processing resources 202 to simulate a game. This game may be a sporting game, such as a tennis game, basketball game, baseball game, softball game, hockey game, football game, soccer game, fencing game, a race, another type of sporting game, or combinations thereof. Other types of games that may be simulated with the game simulator 226 include war games, hunting games, fighting games, other types of games, or combinations thereof.

The training simulator 228 represents programmed instructions that, when executed, cause the processing resources 202 to simulate a situation for training purposes. For example, these situations may include hostage situations for law enforcement personnel, firefighting situations for rescue personnel, disaster situations for rescue personnel, other types of training, or combinations thereof.

The haptic response generator 230 represents programmed instructions that, when executed, cause the processing resources 202 to generate a haptic response. These haptic responses may include using an electrode incorporated into the garment 102 to cause the user to feel a sensation during a workout or a simulation. The reason for generating the haptic response may include generating the haptic response due to over exertion, as a warning, in response to a health condition, to assist with a workout, for another reason, or combinations thereof. In some situations, the haptic response may occur as a result of the user receiving a wound during a training simulation, contacting a virtual ball during a game simulation, contacting a virtual obstacle during a game or training simulation, or combinations thereof.

The garment sensor receiver 232 represents programmed instructions that, when executed, cause the processing resources 202 to receive data from the sensors incorporated into the garment 102. In these examples, the physiological conditions of the user may be determined based on the received data. In some examples, the haptic response generator 230 generates the haptic response based on the received information.

The force determiner 234 represents programmed instructions that, when executed, cause the processing resources 202 to determine the amount of force generated by the user during his or her workout. The movement of the user, the weight lifted by the user, the time that it takes to move the weight, and other factors may be considered when determining how much force is generated by the user.

The location determiner 236 represents programmed instructions that, when executed, cause the processing resources 202 to determine the location of the user. In some situations, the location determiner 236 determines the location based on a global positioning mechanism or another type of locating mechanism incorporated into the garment 102. The user's location over time can be used to determine a distance traveled by the user, the speed of the user, other parameters about the user's route, such as weather, altitude, air pressure, other conditions, or combinations thereof.

Further, the memory resources 204 may be part of an installation package. In response to installing the installation package, the programmed instructions of the memory resources 204 may be downloaded from the installation package's source, such as a portable medium, a server, a remote network location, another location, or combinations thereof. Portable memory media that are compatible with the principles described herein include DVDs, CDs, flash memory, portable disks, magnetic disks, optical disks, other forms of portable memory, or combinations thereof. In other examples, the program instructions are already installed. Here, the memory resources 204 can include integrated memory such as a hard drive, a solid state hard drive, or the like.

In some examples, the processing resources 202 and the memory resources 204 are located within the garment 102, the mobile device 108, the activity information devices 284, another type of device, or combinations thereof. The memory resources 204 may be part of any of these device's main memory, caches, registers, non-volatile memory, or elsewhere in their memory hierarchy. Alternatively, the memory resources 204 may be in communication with the processing resources 202 over a network. Further, the data structures, such as the libraries, may be accessed from a remote location over a network connection while the programmed instructions are located locally. Thus, the system 200 may be implemented on the garment 102, the fitness tracking devices 284, a user device, a mobile device 108, a phone, an electronic tablet, a wearable computing device, a head mounted device, a server, a collection of servers, a networked device, a watch, or combinations thereof. These implementation may occur through input mechanisms, such as push buttons, touch screen buttons, voice commands, dials, levers, other types of input mechanisms, or combinations thereof. Any appropriate type of wearable device may include, but are not limited to glasses, arm bands, leg bands, torso bands, head bands, chest straps, wrist watches, belts, earrings, nose rings, other types of ring, necklaces, garment integrated devices, other types of devices, or combinations thereof.

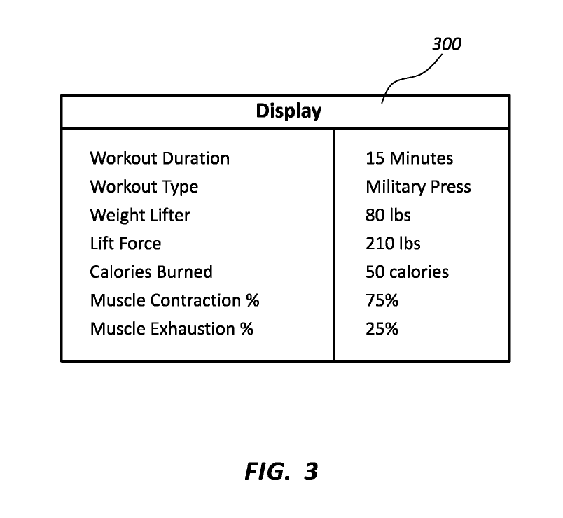

FIG. 3 illustrates a view of an example of a display 300 incorporated into a computing device in accordance with the present disclosure. In this example, the display 300 include information about the user's workout. The computing device may be part of the activity information device, the mobile device, a desktop, a laptop, a digital device, a watch, a wearable computing device, glasses, another type of device, or combinations thereof.

In the example of FIG. 3, the display 300 depicts that the duration of the workout so far has been 15 minutes and that the user is currently performing a military press type workout. The amount of weight being lifted by the user is 80 pounds and the user is lifting the weight with a force of 210 pounds. This force may be determined, in part, by the speed at which the user lifts the weight. Further, the display 300 depicts, based on the characteristics of the workout so far, the user has already burned 50 calories. Also, during the current lift, the user's muscle contraction is 75 percent and the muscles' total exhaustion is 25 percent.

The muscle contraction percentages may be based, in part, on the strength of the electrical signal detected by the surface electromyography sensor. This percentage may be presented to the user so the user understands how much more energy he or she may be capable to exerting to maximize his or her workout per lift. The muscle exhaustion reading may indicate to the user how much more overall capability a muscle group has available. The number may be based in part on electrical signals detected with the electromyography sensor, the movement of the user's body parts associated with the lift detected by an accelerometer, other detected parameters of the user's workout, or combinations thereof.

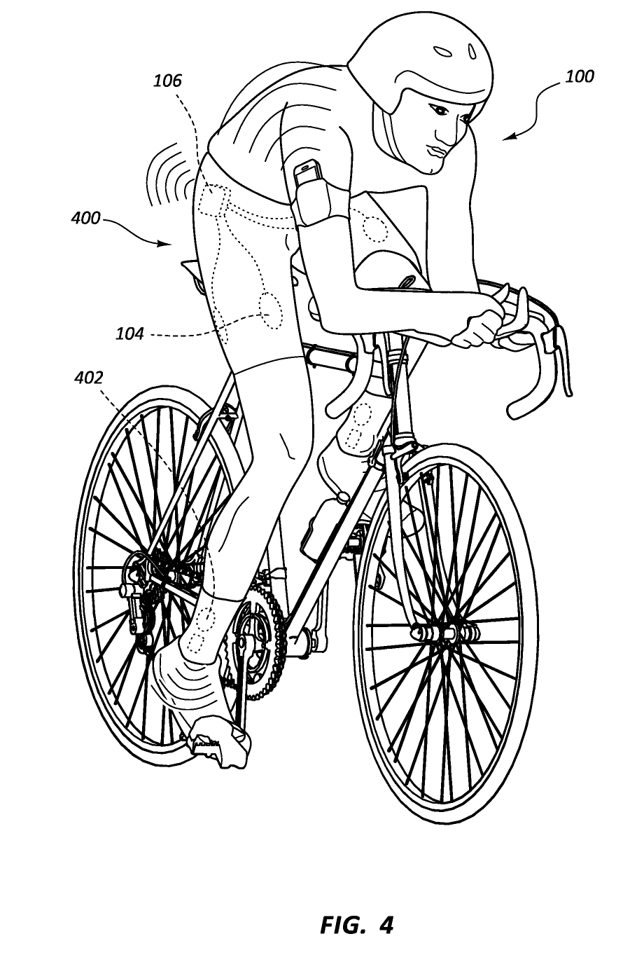

FIG. 4 illustrates a perspective view of an example of a garment 102 tracking a user activity in accordance with the present disclosure. In this example, the user is wearing biking shorts 400 that include multiple sensors 104 for detecting at least one parameter of the user's workout. The sensors 104 of the biking shorts 400 may include electromyography sensors to detect muscle activity, accelerometers to detect leg movement, or other parameters. In some examples, accelerometers incorporated into the biking shorts 400 may have a capability of distinguishing between different movement directions and/or types of movement. For example, the accelerometers may have the ability to detect movement that is associated with changes in the direction that the user is traveling on the bicycle as well as detect leg movement associated with pedaling the bicycle.

Also, in the example of FIG. 4, the user is wearing socks 402 with at least one sensor 104. Likewise, the sensor 104 may include an electromyography sensor, an accelerometer, another type of sensor configured to detect a parameter of the user's workout, or combinations thereof. In some examples, data collected from the socks and biking shorts may be collectively used to determine attributes of the user's workout. In some examples, the user's shirt and/or other clothing may also incorporate sensors. Each of the sensors and/or garments with sensors may communicate with at least one wireless device. The wireless device may be the activity information device or the wireless device may be in communication with the activity information device. In the example of FIG. 4, the wireless device is a mobile device 108 being worn by the user. In some examples, the wireless device may be incorporated into the bicycle.

At least one mechanism incorporated into the biking shorts 400 and/or socks 402 may apply a haptic response to the user in response to a warning about a workout and/or physiological condition or the user. In other examples, the haptic response may be generated to assist the user with his or her workout.

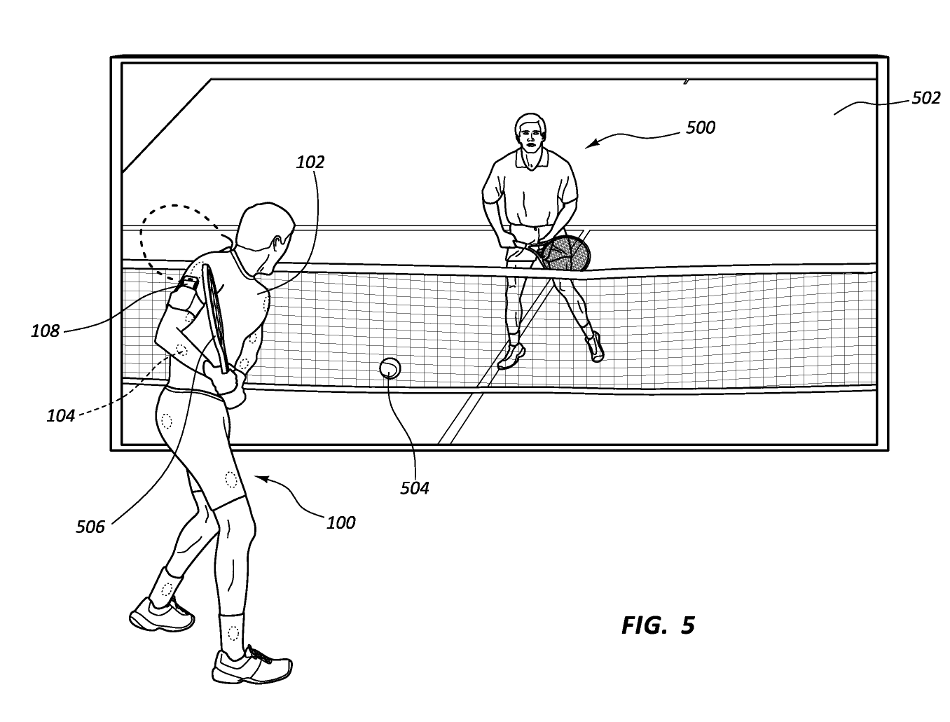

FIG. 5 illustrates a perspective view of an example of a garment 102 tracking a user activity in accordance with the present disclosure. In this example, the user is playing a virtual game of tennis with a virtual player 500 depicted in a screen 502. The game includes a virtual tennis ball 504. The user may wear garments 102, such as shirts, shorts, socks, or other types of clothing that incorporate sensors that record parameters of the user's workout and/or have the capability of applying a haptic response to the user. In some examples, a haptic response may be applied to the user if the virtual tennis ball 504 hits the user. In some examples, the haptic response may be generated in response to the user hitting the virtual tennis ball 504 with the tennis racket 506 to simulate what the user would feel in his or her arm or other parts of his or her body if the user actually hit a real tennis ball.

While this example has been depicted with a user playing a virtual game of tennis, any appropriate type of simulated game, simulated training, other simulated experience, or combinations thereof may be used in accordance with the principles described in the present disclosure. For example, these principles may be applied to simulated soccer games, baseball games, martial arts games, basketball games, dodge ball games, car racing games, professional simulations, law enforcement simulations, military simulations, hunting simulations, other types of simulations, or combinations thereof.

In other examples, the haptic response may be used to communicate a message to the user, such as a physiological condition. In other examples, the message may be a game related message, such as a message indicating the conclusion of the game, a time warning, a penalty, a score achievement, another type of game related message, or combinations thereof.

While the examples above have been described with reference to an electrode providing a haptic response, any appropriate type of mechanism may be used to provide a haptic response to the user in accordance with the principles described herein. For example, a vibrator may be used to apply a localized vibration to the user. In other examples, the temperature device may be used to apply the haptic response. In this example, the haptic response may include localized heating or cooling of the user's skin to communicate a message to the user. In other examples, a haptic response generator may include an ability to compress the user's skin, stretch the user's skin, twist the user's skin, apply pressure to the user's skin, apply an electric stimulation to the user's skin, apply a magnetic stimulation to the user's skin, move air or another type of gas across the user's skin, apply a shear force across the user's skin, apply another type of sensation to the user's skin, or combinations thereof.

### INDUSTRIAL APPLICABILITY

In general, the invention disclosed herein may provide the user with an ability to have his or his activity tracked by a garment that is also capable of delivering messages to the user. The messages may indicate to the user that he or she has a physiological condition. In other examples, these message may indicate a parameter about a simulated experience in which the user is participating. For example, the haptic response may indicate that the user has come into contact with a virtual object, such as a virtual obstacle, a virtual ball, a virtual opponent, another virtual object, or combinations thereof.

The garment may include at least one sensor that is capable of collecting data about at least one parameter of the user, such as the user's activity, a health condition, a physiological condition, an environment condition in which the user is located, another type of condition, or combinations thereof. In some examples, the haptic response is delivered to the user based in part on processing performed on the collected data. For example, if the collected data indicates a particular condition, a haptic response may be generated to communicate the condition to the user. A non-exhaustive list of the conditions includes that the user's heart rate is too high, a muscle group of the user is too fatigued, a respiration rate of the user is too high, a calorie burn goal has been reached, a force generated by the user, determine a location of the user, or another condition.

The sensors may be incorporated into the user's garments so that the sensors are appropriately located to sense conditions or deliver messages to the user. For example, an electromyography sensor may be placed proximate each muscle or muscle group of interest. Likewise, accelerometers may be placed on limbs or other body parts that move to aid in collecting the types of data that can be used to determine the conditions of the user. Similarly, other types of sensors may be appropriately positioned about the appropriate parts of the user's body to collect information. For example, the electrode may be positioned proximate a forearm muscle, a bicep muscle, a shoulder muscle, a tricep muscle, a quadriceps muscle, a hamstring muscle, an adductor muscle, a calf muscle, a trapezius muscle, a latissimus dorsi muscle, a pectoralis muscle, an abdominal muscle, an oblique muscle, another type of muscle, or combinations thereof.

In some cases, the fabric of the garment is a compressive fabric that is constructed to hold the sensors and/or haptic response mechanism close to the user's skin. While the above examples have been described with reference to garments made of compressive fabrics, any appropriate type of fabric may be used in accordance with the principles described herein.

In some examples, the electrode, accelerometer, other type of sensor, haptic response mechanism, wireless device, or other type of device is interwoven into the fabric of the garment. In other examples, these devices are secured to the garment through a strap or another type of attachment device. In some examples, these devices are disposed within pockets formed in the garment.

The sensors may be in communication with an activity information device. This activity information device may be capable of tracking activity information, fitness information, physiological information, or other types of data about the user. The information may be stored and tracked over time, such as over the course of multiple workouts, multiple days, multiple months, multiple years, other time periods, or combinations thereof. Further, The activity information device may be used to determine parameters about the user's workout. This activity information device may also generate the haptic response to send the user an appropriate message.

The activity information device may also simulate an experience for the user. For example, this experience may include a simulated route on which the user can run, bike, row, or perform another type of aerobic exercise. In some examples, the simulated experience is a sporting game, a training simulation, another type of simulation, or combinations thereof. The activity information device may communicate with the garment to generate a haptic response to be part of the simulation.

The sensors may include at least one electrode that is capable of detecting at least one characteristic of the user. For example, one or more electrodes may be positioned adjacent regions of the user's body through the garment to receive electrical cardio signals of the user. These electrical cardio signals can be used to determine the user's heart rate.

In another example, an electrode may be positioned to receive electromyography signals that detect muscle contraction. For example, the sensors may be positioned over each muscle group of interest. In some examples, sensors are positioned over the user's deltoid muscles, bicep muscles, and forearm muscles. But, the surface electromyography sensors can be positioned proximate pectoral muscles, trapezius muscles, oblique muscles, abdominal muscles, latissimus dorsi muscles, tricep muscles, hamstring muscles, quadriceps muscles, calf muscles, adductor muscles, other types of muscles, or combinations thereof. As the muscles contract, the corresponding electromyography sensor may detect an electrical signal indicating the muscle contraction. In some examples, the electromyography sensor can also detect the contraction percentage of the muscle. The contraction percentage can indicate to the user how effectively he or she is working out his or her muscles.

The garment may also include accelerometers. These accelerometers may be incorporated into the garment in any appropriate location to determine the types of body movements performed by the user. For example, a three axis accelerometer may be incorporated into the garment to determine vertical and horizontal movements. The movement patterns can be analyzed to determine the user's types of movements. For example, jumping movements may exhibit different types of patterns than walking movements. Further, one or more accelerometers may be incorporated into each sleeve of the garment. For example, an accelerometer incorporated into the upper portion of the sleeve can detect when the upper arm moves, and another accelerometer incorporated into the lower sleeve can detect when the lower arm moves. With this arrangement, complex arm movements can be detected. Similarly, other arrangements with multiple accelerometers can determine whether the user is jumping, twisting, curling, walking, running, performing another type of movement, or combinations thereof.

Accelerometers may also be used to determine a respiration count of the user. For example, at least one accelerometer positioned about the user's chest can be used to determine when the user's chest expands and contracts in accordance with the user's breathing. In other examples, a strain gauge may be incorporated into the garment, and as the user's chest expands from breathing, the strain gauge stretches. As the strain gauge stretches, it generates a signal that can be sent to the activity information device.

Such recorded movements, in conjunction with recorded muscle contractions, provide a degree of detail about the user's workout. These details can be analyzed to determine the force exerted by the user during the workout, the number of calories burned by the user during the workout, other types of details about the workout, or combinations thereof.

In some examples, the garment includes a location determining device. The device may be a global positioning device, another type of location determining, or combinations thereof. In some examples, the location determining device may be used to determine a distance that a user has traveled, a speed at which the user has traveled, an altitude at which the user is at, an altitude that the user has traveled, other parameters, or combinations thereof.

Each sensor may be in communication with the wireless device. In some examples, each sensor is in communication with the wireless device through an independent electrically conductive medium. In other examples, the sensors communicate with each other and communicate with the wireless device through other sensors. For example, the wireless device may be in direct communication with a first sensor and indirectly in communication with a second sensor through the first sensor. The second sensor can send information towards the wireless device by sending the information to the first sensor, which then sends the information on to the wireless device. In this example, the sensors form a network. The sensor network may allow sensors to communicate with each other. In some examples, the communications are bidirectional where the first sensor can send messages to the second sensor and the second sensor can send messages to the first sensor. The networks may have any appropriate network topology, such as a daisy chain topology, a bus topology, a star topology, a mesh topology, ring topology, a tree topology, a linear topology, a fully connected topology, another type of topology, or combinations thereof.

An electrically conductive medium may include a cable or another type of wire that is disposed within channels formed within the fabric of the garment. In other examples, an electrically conductive thread is used to create an electrically conductive pathway formed in the fabric of the garment. For example, a single thread may be used to create the electrically conductive pathway. In other examples, multiple threads are used to form a patch of electrically conductive fabric capable of conducting an electrical signal. The electrically conductive fabric may be covered by an outer fabric layer, an inner fabric layer, a waterproof layer, a breathable layer, another type of layer, or combinations thereof. In some examples, an electrically conductive fabric is exposed in the inner or outer surfaces of the garment.

Any appropriate communication mechanisms may be used to enable communication between the components of the garment. For example, the sensors may be in communication with each other through fiber optic cables, wireless transceivers, other types of communication channels, or combinations thereof. In some examples, the garment includes multiple wireless devices that are capable of communication with the activity information device directly or indirectly.

In some examples, the activity information device is caused to record and store the information received from the garment by an activity tracking program. One type of activity tracking program that may be compatible with the principles described in the present disclosure is the iFit program which is accessible through website www.ifit.com (last visited May 19, 2014) and administered through ICON Health and Fitness, Inc. headquartered in Logan, Utah. The activity tracking program may calculate the number of calories burned by the user, track the amount of weight lifted by the user, track the number of sets and/or repetitions lifted by the user, determine the heart rate of the user, determine the respiration of the user, track the duration of the user's workout, record other types of information, or combinations thereof. In some examples, the user has access to the information about the workout in real time. In these examples, the information may be presented to the user, friend, trainer, or another type of individual to the user through a screen of the mobile device or a larger screen that is easy/convenient for the user to view while working out. Further, this information may be available to the user after the workout is over. In some examples, the user may access the information over the internet. In other examples, the user may access the information through the local memory of the mobile device, laptop, desktop, or other digital device that contains the information. The information may also be available to other users of the activity tracking program. In these situations, the user may be in a competition with other users of the activity tracking program, or the user may allow a trainer, health professional, or other type of user to review the information.

In some examples, the activity tracking program gives the user advice. For example, if the user indicates that the user desires to achieve certain fitness goals within a predetermined time period, the activity tracking program may provide feedback to the user about his or her progress towards to the fitness goals. In some examples, the activity tracking program may give recommendations to the user to help assist the user towards his or her goals. These recommendations may include recommending that the user lift heavier weights for certain types of lifts, run at a faster pace, exercise for a longer period of time, other types of recommendations, or combinations thereof. In some examples, these recommendations may include safety recommendations, such as recommending to the user to slow down, lift lighter weights for specific lifts, other types of recommendations, or combinations thereof. These recommendations may be based in part of the user's heart rate, desired goals, the muscle percentage contraction, other types of information, or combinations thereof. While this example has been described as being compatible with a specific activity tracking program with specific features, any appropriate type of activity tracking program and/or features of the program may be used in accordance with the principles described herein.

In addition to collecting information about the user's activities and resulting physiological conditions, the sensors may be configured to provide a haptic response to the user. These haptic responses may be generated for any appropriate type of reason. For example, a haptic response may be provided to indicate to the user that he or she is over exerting himself or herself. This haptic response may be generated by applying a safe localized stimulus to the user. This electrode may be used to gather specific types of data in addition to generating haptic responses. But, in other examples, this electrode may be dedicated to providing haptic responses. Haptic responses that are generated as a warning to the user may be accompanied with warnings displayed in the mobile device, an audible warning, or another type of warning mechanism. But, in some examples, the haptic response is the sole mechanism for warning the user about physiological condition.

The haptic response may also be administered as part of a simulation. For example, if the user is performing a workout that is simulated to be in a particular environment, such as a forest, and the user runs into a virtual obstacle, such as a tree, a haptic response may be administered to provide a realistic element to the simulation.

In other examples, a haptic response may be administered to assist the user in performing a lift. In some circumstances, an appropriately applied electrical pulse from an electrode may cause or increase a muscle contraction. Thus, if the user is struggling to lift a weight, a haptic response may be applied to cause the user's muscles to contract to complete the lift. The assisted contractions may contribute to increased muscles simulation and growth. In some examples, the user may have an option to have the haptic response assistance just when the user is struggling, for every lift, or not at all.

Further, while the above examples have been described with reference to performing calculations and other forms of interpreting the data collected by the sensors with the activity information device, any appropriate location for performing the calculations and/or interpretations may be used in accordance with the principles described in the present disclosure. For example, this processing may occur on the mobile device, a networked device, a computing device incorporated into the garment, another type of device, or combinations thereof.

In some examples, a battery or another type of power source is incorporated into the garment. This battery may be a disposable battery or a rechargeable battery. In some cases, the garment may include an energy harvesting mechanism, such as a linear generator that can harvest the movements of the user to produce energy or a thermoelectric device that can use the thermal differential between the user's body heat and the ambient temperature of the air surrounding the user to provide energy to power the sensors of the garment. In some examples, the energy harvesting mechanisms supplement the battery or other power source in the garment or the energy harvesting mechanism can be used to recharge the batteries.

While the examples are described above with reference to the haptic input mechanism including an electrode, the haptic input mechanism may include any appropriate type of devices. For example, the haptic input response may include an eccentric weight attached to a motor. In this example, when the motor is activated, the motor causes the eccentric weight to rotor off balance, which creates a vibration. These motors and eccentric weights may be incorporated into the garment's fabric.

What is claimed is:

1. A garment, comprising:
fabric;
at least one electrode attached to the fabric;
at least one accelerometer attached to the fabric;
a wireless device in communication with an activity information device; and
at least one haptic input mechanism incorporated into the fabric that provides a haptic response in response to communications with the activity information device.

2. The garment of claim 1, wherein the activity information device is a fitness tracking device.

3. The garment of claim 1, wherein the activity information device is an environment simulation device.

4. The garment of claim 3, wherein the environment simulation device is programmed to cause an aerobic exercise device to simulate a real world route.

5. The garment of claim 3, wherein the environment simulation device is programmed to simulate a sporting event.

6. The garment of claim 1, wherein the at least one electrode is part of a surface electromyography system.

7. The garment of claim 1, wherein the at least one electrode is interwoven into the fabric.

8. The garment of claim 1, wherein an accelerometer is interwoven into the fabric.

9. The garment of claim 1, wherein the at least one electrode measures muscle activity of a user wearing the garment when the garment is worn by the user.

10. The garment of claim 1, wherein the at least one electrode includes multiple electrodes positioned to measure activity of at least two of a forearm muscle, a bicep muscle, a shoulder muscle, and a tricep muscle.

11. The garment of claim 1, wherein the at least one electrode includes multiple electrodes positioned to measure activity of at least two of a quadriceps muscle, a hamstring muscle, an adductor muscle, and a calf muscle.

12. The garment of claim 1, wherein the at least one electrode includes multiple electrodes positioned to measure activity of at least one of a shoulder muscle, a trapezius muscle, and a latissimus dorsi muscle.

13. The garment of claim 1, wherein the at least one electrode includes multiple electrodes positioned to measure activity of at least one of a pectoralis muscle, an abdominal muscle, and an oblique muscle.

14. The garment of claim 1, wherein the activity information device determines a force generated by a user during a workout.

15. The garment of claim 1, wherein the haptic input mechanism includes an eccentric weight and a motor.

16. A garment, comprising:
fabric;
at least one electrode interwoven into the fabric where the at least one electrode is part of a surface electromyography system;
at least one accelerometer attached to the fabric; and
a wireless device in communication with an environment simulation device;
wherein the at least one electrode provides a haptic response based on communications with the environment simulation device.

17. The garment of claim 156, wherein the environment simulation device is also a fitness tracking device.

18. The garment of claim 17, wherein the fitness tracking device determines a number of calories burned by a user during a workout.

19. The garment of claim 16, wherein the garment is a sock.

20. A garment, comprising:
compressive fabric;
at least one electrode interwoven into the compressive fabric where the at least one electrode is part of a surface electromyography system;
at least one accelerometer attached to the compressive fabric; and
a wireless device in communication with a device that simulates a workout environment and tracks fitness of a user;
wherein the at least one electrode provides a haptic response based on user interaction with a simulated object from the workout environment with the device, and wherein the wireless device determines a force generated by the user during a workout and determines a number of calories burned by the user during the workout.
